(19) United States
(12) Patent Application Publication
(10) Pub. No.: US 2026/0090910 A1
(43) Pub. Date: Apr. 2, 2026

(54) HEATING GARMENT

(71) Applicant: REB LLC, McMinnville, OR (US)

(72) Inventor: Katelyn BRAY, McMinnville, OR (US)

(73) Assignee: REB LLC, McMinnville, OR (US)

(21) Appl. No.: 18/899,652

(22) Filed: Sep. 27, 2024

(51) Int. Cl. A61F 7/02; A61F 7/00

(52) U.S. Cl. CPC A61F 7/02; A61F 7/007; A61F 2007/005; A61F 2007/0231; A61F 2007/0236

(57) ABSTRACT

A heating garment includes a first piece of material, a second piece of material, a plurality of heating elements, and a plurality of coupling mechanisms. A pocket is defined between the first and second pieces of material. The plurality of heating elements is disposed in the pocket defined between the first piece of material and the second piece of material. The plurality of heating elements is configured to run from a top of the heating garment to a bottom of the heating garment. The plurality of coupling mechanisms is formed on the heating garment. The plurality of coupling mechanisms includes a first coupling mechanism formed on a first external surface of the first piece of material, a second coupling mechanism formed on a second external surface of the second piece of material, and a third coupling mechanism formed on the second external surface of the second piece of material.

### FIELD OF DISCLOSURE

The present disclosure generally relates to a heating apparatus and more specifically a heating garment designed to address and relieve menstrual pain.

### BACKGROUND

Heating pads typically operate by allowing a user to increase or decrease an amount of current provided to the heating pad's heating elements in order to increase or decrease the operating temperature of the heating pad. Heating pads assist users in relieving their pain by promoting circulation in the area to which the heating pad is applied.

### SUMMARY

In some embodiments, a heating garment is disclosed herein. The heating garment comprises a first piece of material, a second piece of material, a plurality of heating elements, and a plurality of coupling mechanisms. The first piece of material is coupled with the second piece of material. A pocket is defined between the first piece of material and the second piece of material. The plurality of heating elements is disposed in the pocket defined between the first piece of material and the second piece of material. The plurality of heating elements is configured to run from a top of the heating garment to a bottom of the heating garment. The plurality of coupling mechanisms is formed on the heating garment. The plurality of coupling mechanisms comprises a first coupling mechanism formed on a first external surface of the first piece of material, a second coupling mechanism formed on a second external surface of the second piece of material, and a third coupling mechanism formed on the second external surface of the second piece of material. The heating garment is configured to operate between an uncoupled mode and a coupled mode. In the coupled mode, the second coupling mechanism and the third coupling mechanism are configured to mate with the first coupling mechanism, forming a torso opening and two leg openings in the heating garment.

In some embodiments, a heating system is disclosed herein. The heating system includes a heating garment and a controller. The heating garment comprises a first piece of material, a second piece of material, a plurality of heating elements, and a plurality of coupling mechanisms. The first piece of material is coupled with the second piece of material. A pocket is defined between the first piece of material and the second piece of material. The plurality of heating elements is disposed in the pocket defined between the first piece of material and the second piece of material. The plurality of heating elements is configured to run from a top of the heating garment to a bottom of the heating garment. The plurality of coupling mechanisms is formed on the heating garment. The plurality of coupling mechanisms comprises a first coupling mechanism formed on a first external surface of the first piece of material, a second coupling mechanism formed on a second external surface of the second piece of material, and a third coupling mechanism formed on the second external surface of the second piece of material. The heating garment is configured to operate between an uncoupled mode and a coupled mode. In the coupled mode, the second coupling mechanism and the third coupling mechanism are configured to mate with the first coupling mechanism, forming a torso opening and two leg openings in the heating garment. The controller is in communication with the heating garment. The controller is configured to control an operating temperature of the heating garment.

### BRIEF DESCRIPTION OF THE DRAWINGS

The accompanying drawings, which are incorporated herein and form part of the specification, illustrate the present disclosure and, together with the description, further serve to explain the principles of the present disclosure and to enable a person skilled in the relevant art(s) to make and use embodiments described herein.

FIG. 1A represents a bottom view of a heating garment, according to example embodiments.

FIG. 1B represents a top view of the heating garment shown in FIG. 1A, according to example embodiments.

FIG. 1C illustrates a rear side view of the heating garment of FIGS. 1A-1B in an uncoupled, folded form, according to example embodiments.

FIG. 1D illustrates a rear side view of the heating garment of FIGS. 1A-1C in a coupled, folded form, according to example embodiments.

FIG. 1E illustrates a front side view of the heating garment of FIGS. 1A-1D in an uncoupled, folded form, according to example embodiments.

FIG. 1F illustrates a front side view of the heating garment of FIGS. 1A-1E in a coupled, folded form, according to example embodiments.

FIG. 2A is a partial cut-away top view of the heating garment of FIGS. 1A-1F, according to example embodiments.

FIG. 2B is a full cut-away top view of the heating element of FIGS. 1A-1F and FIG. 2A, according to example embodiments.

FIG. 3 represents a top view of the heating garment shown in FIGS. 1A-1F, according to example embodiments.

FIG. 4A is a block diagram illustrating an operating environment, according to example embodiments.

FIG. 4B is a block diagram illustrating an operating environment, according to example embodiments.

FIG. 4C is a block diagram illustrating an operating environment, according to example embodiments.

FIG. 5A is a block diagram illustrating a computing device, according to example embodiments of the present disclosure.

FIG. 5B is a block diagram illustrating a computing device, according to example embodiments of the present disclosure.

The features of the present disclosure will become more apparent from the detailed description set forth below when taken in conjunction with the drawings, in which like reference characters identify corresponding elements throughout. In the drawings, like reference numbers generally indicate identical, functionally similar, and/or structurally similar elements. Additionally, generally, the left-most digit(s) of a reference number identifies the drawing in which the reference number first appears. Unless otherwise indicated, the drawings provided throughout the disclosure should not be interpreted as to-scale drawings.

### DETAILED DESCRIPTION

Menstrual cramps, or dysmenorrhea, affect a significant portion of the female population and often leads to discomfort and impaired daily functioning. Traditional methods to alleviate menstrual pain typically include oral medications, such as nonsteroidal anti-inflammatory drugs, and the application of heat. Heating pads are widely used as a means to provide relief by relaxing the muscles and improving blood flow to an affected area. However, conventional heating pad designs are typically lacking in their effectiveness at addressing the specific problem of menstrual pain. For example, conventional heating pads are typically not designed for mobility or continuous use through daily activity. Conventional heating pads also do not include a robust array of heating elements to ensure full application of heat throughout the heating pad. As a result, there is a need in the industry for an improved heating pad garment, specifically tailored for relieving menstrual pain.

One or more techniques described herein provide an improved heating apparatus designed to address menstrual pain. For example, one or more techniques described herein provide a heating pad that takes the form of an underwear-like garment that may be worn to relieve menstrual pain. To improve upon conventional heating pads, the present design provides an easy to wear apparatus that provides improvements in both mobility and continuous use. Further, due to the robust heating element array disposed in the heating garment, the present apparatus is able to provide wearers with a more robust solution that is able to simultaneously heat the groin, lower back, hips, and front lower abdomen.

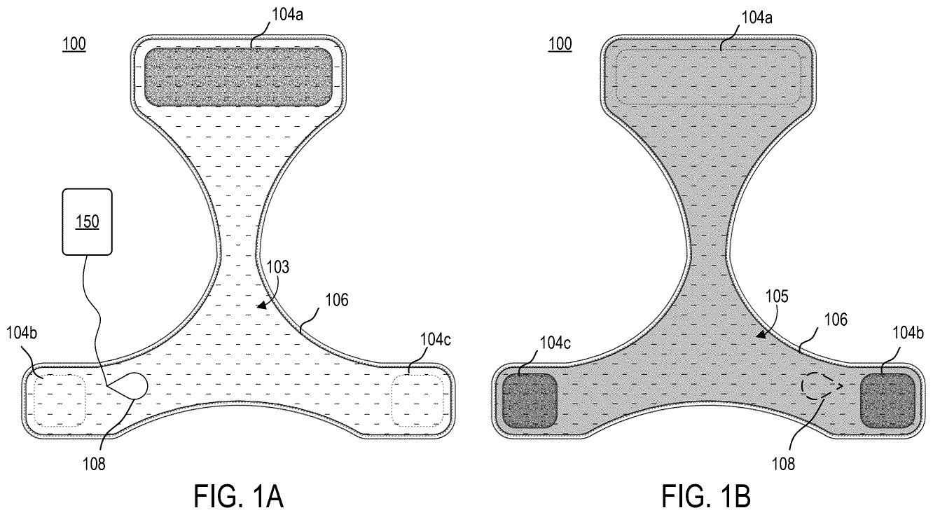

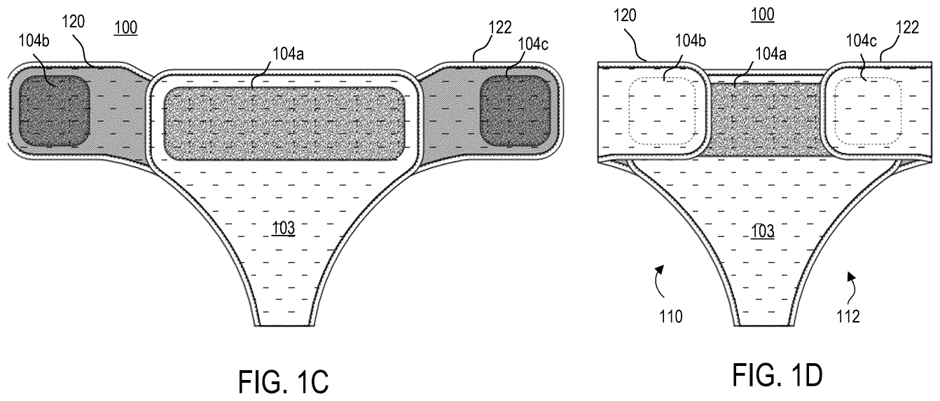

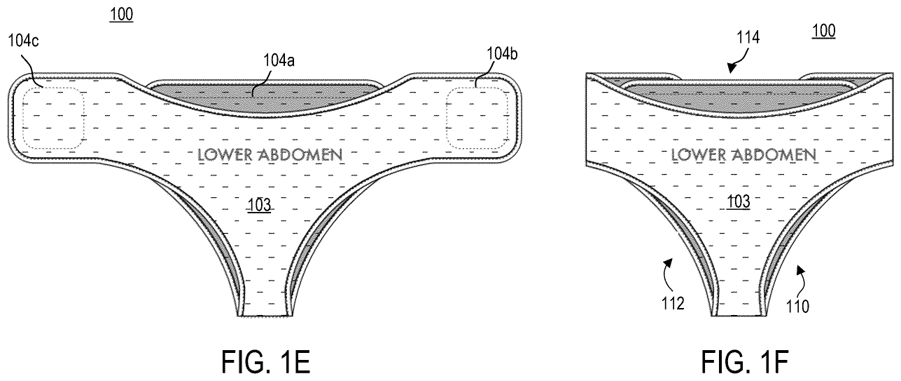

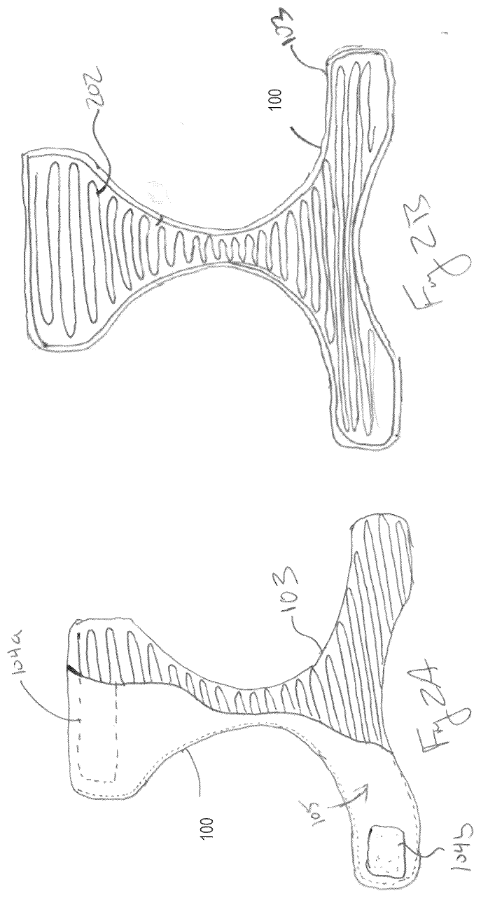

FIGS. 1A-1F illustrate various views a heating garment 100, according to example embodiments. For example, FIG. 1A represents a bottom view of heating garment 100 and FIG. 1B represents a top view of heating garment 100, according to example embodiments. As shown, heating garment 100 may be representative of a garment configured to be worn like underwear or a bikini bottom.

Heating garment 100 may include a first piece of material 103 and a second piece of material 105. First piece of material 103 may be coupled with second piece of material 105 via one or more welds 106. For example, first piece of material 103 and second piece of material 105 may be stitched together to form a cohesive garment. Heating garment 100 may include a heating system (not fully shown) at least partially disposed between first piece of material 103 and second piece of material 105. For example, the heating system may include a plurality of heating elements enclosed between first piece of material 103 and second piece of material 105. In some embodiments, the plurality of heating elements may be formed or enclosed by a water-resistant material in order to protect the heating elements from potential damage.

Heating garment 100 may further include coupling mechanisms 104a-104c formed thereon. In some embodiments, coupling mechanism 104a may be formed on a first surface of heating garment 100. For example, as shown, coupling mechanism 104a may be formed on first piece of material 103. In some embodiments, coupling mechanism 104b and coupling mechanism 104c may be formed on a second surface of the heating garment, opposite the first surface where coupling mechanism 104a may be formed. For example, as shown, coupling mechanism 104b and coupling mechanism 104c may be formed on second piece of material 105. In operation, coupling mechanism 104b and coupling mechanism 104c may be configured to mate or couple with coupling mechanism 104a. Using a non-limiting example, such as when coupling mechanisms 104a-c are realized using VELCRO®, coupling mechanism 104a may be the “soft side” (e.g., loop side) and coupling mechanisms 104b-c may be the rough side (e.g., hook side). It is important to note that VELCRO® and similar products are only one of a plurality of possible coupling mechanisms that may be utilized.

Heating garment 100 may further include a connection element 108. Connection element 108 may be a component of the heating system. Connection element 108 may be configured to be in electrical communication with heating elements disposed between first piece of material 103 and second piece of material 105. In operation, connection element 108 may be configured to interface with a controller 150. For example, connection element 108 may include an input port with which controller 150 may mate. When connected, controller 150 may be used to control the heating elements disposed in heating garment 100. For example, controller 150 may increase or decrease the operating temperature by increasing or decreasing the amount of current flowing to the heating elements. In this manner, connection element 108 may provide an electrical interface between controller 150 and the plurality of heating elements.

Controller 150 may be representative of a standard wired or wireless controller configured to control the heating elements disposed in heating garment 100. In some embodiments, such as those discussed above, controller 150 may be configured to interface with heating elements through a direct physical connection via connection element 108. In some embodiments, controller 150 may be configured to interface with heating elements through an indirect or remote connection, such as through one or more communication protocols, such as, but not limited to: Bluetooth, WiFi, Zigbee, RFID, NFC, Z-wave, and the like.

Generally, controller 150 may be configured to have a power source. In some embodiments, such as when controller 150 is physically coupled with heating garment 100 via connection element 108, controller 150 may have a corded power source (e.g., two- or three-pronged cord configured to plug into an outlet). In some embodiments, such as when controller 150 is coupled with heating garment 100 through non-contact means (e.g., Bluetooth, WiFi, Zigbee, RFID, NFC, Z-wave, etc.), controller 150 and connection element 108 may be separately powered via battery means.

In some embodiments, controller 150 may include an interface. The interface may include one or more actionable elements to control operation of the heating garment. Exemplary actionable elements may include, but are not limited to, a first actionable element corresponding to a power button which powers the heating garment on or off, a second actionable element corresponding to an increase temperature button, which causes the heating garment to increase its operating temperature by supplying the heating elements with additional current, a third actionable element corresponding to a decrease temperature button, which causes the heating garment to decrease its operating temperature by supplying the heating elements with less current, and/or a fourth actionable element corresponding to a set button, which is configured to toggle between one or more set timers for operation. Although four actionable elements are described, those skilled in the art understand that fewer actionable elements or more actionable elements may be present on controller 150.

In some embodiments, via controller 150, a user may instruct the heating garment to continue to operate for a plurality of preset intervals of time (e.g., one hour, six hours, twelve hours, twenty-four hours, and the like).

In some embodiments, controller 150 may further be configured to display the operating temperature of the heating garment. In this manner, a user may be able to set a desired operating temperature to be maintained by controller 150.

In some embodiments, heating garment 100 may include one or more pockets or pouches formed in one or more of first piece of material 103 or second piece of material 105. These one or more pockets or pouches may allow for the inclusion of additional material such that heating garment 100 may act as a weighted garment to place additional pressure on the locations of which heating garment 100 may be worn. In some embodiments, heating garment 100 may include one or more layers of padding material between first piece of material 103 and second piece of material 105 to protect the wearer from direct or near direct contact with the heating elements.

FIGS. 1C-1D are rear side views of heating garment 100, according to example embodiments. As shown, FIG. 1C illustrates heating garment 100 in an uncoupled, but folded form. For example, as shown, heating garment 100 may be folded at or near a crotch region. FIG. 1D illustrates heating garment 100 in a coupled and folded form. As shown, heating garment 100 when folded near the crotch region and coupled forms an underwear-type garment to be worn by individuals. Heating garment 100 may include wings 120, 122, as part of the cohesive garment that allow for tightening or loosening of heating garment 100 when worn.

FIGS. 1E-1F are front side views of heating garment 100, according to example embodiments. For example, as shown, FIG. 1E illustrates heating garment 100 in an uncoupled, but folded form at or near the crotch area. FIG. 1F illustrates heating garment 100 in a coupled and folded form at or near the crotch area. As shown in FIG. 1F, when heating garment 100 is in a coupled and folded form, openings 110, 112, 114 may be formed. For example, opening 114 may be positioned at or near a wearer's abdomen; openings 110, 112 may be utilized for passage of the wearer's legs when worn.

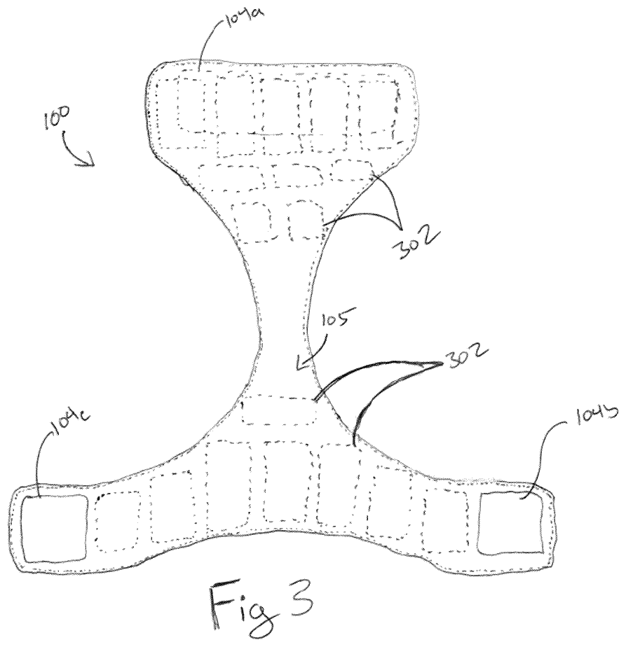

FIG. 2A is a partial cut-away top view of heating garment 100, according to example embodiments. As shown, second piece of material 105 is partially removed to reveal heating elements 202. Heating elements 202 may be disposed in a pocket or opening formed between first piece of material 103 and second piece of material 105. In some embodiments, such as that shown, heating elements 202 may take the form of a serpent design to increase the surface area of heating garment 100 that is heated. As shown, heating elements 202 may be formed throughout heating garment 100. In some embodiments, heating elements 202 may be protected using a waterproof or hydrophobic materials that is placed between heating elements 202 and first piece of material 103 and/or second piece of material 105.

FIG. 2B is a full cut-away top view of heating garment 100, according to example embodiments. To better illustrates heating elements 202, FIG. 2B illustrates heating garment 100 with second piece of material 105 fully removed. As shown, heating elements 202 are disposed throughout heating garment 100—from edge to edge.

The design of heating garment 100 and heating elements 202 disposed therein improve upon conventional heating pad solutions. For example, heating garment 100 may be configured to directly address the source of menstrual pain during menstrual cramping by heating the groin, lower back, hips, and front lower abdomen simultaneously. In this manner, heating garment 100 provides a significant advantage to conventional heating solutions on the market.

Although a specific heating element design is shown, those skilled in the art understand that other heating elements designs may be used provided they are able to heat the groin, lower back, hips, and front lower abdomen areas simultaneously.

FIG. 3 represents a top view of heating garment 100 shown in FIGS. 1A-1F, according to example embodiments. As shown and indicated above, in some embodiments, heating garment 100 may function as a weighted garment to place additional pressure on the locations of which heating garment 100 may be worn. In such embodiments, heating garment 100 may include one or more weights 302 distributed in one or more locations of heating garment 100. In some embodiments, one or more weights 302 may be placed into pockets or openings formed in one of first piece of material 103 or second piece of material 105. In some embodiments, one or more weights 302 may be placed between first piece of material 103 and second piece of material 105. In some embodiments, one or more weights 302 may be removable from heating garment 100 to increase or decrease the overall weight of heating garment 100, such as to conform to the user's preferences. Generally, weights 302 may be formed from any material. In some embodiments, weights 302 may be formed from a heat absorbing material, such that heat generated by heating elements 202 may be retained and/or passed on the wearer of heating garment 100.

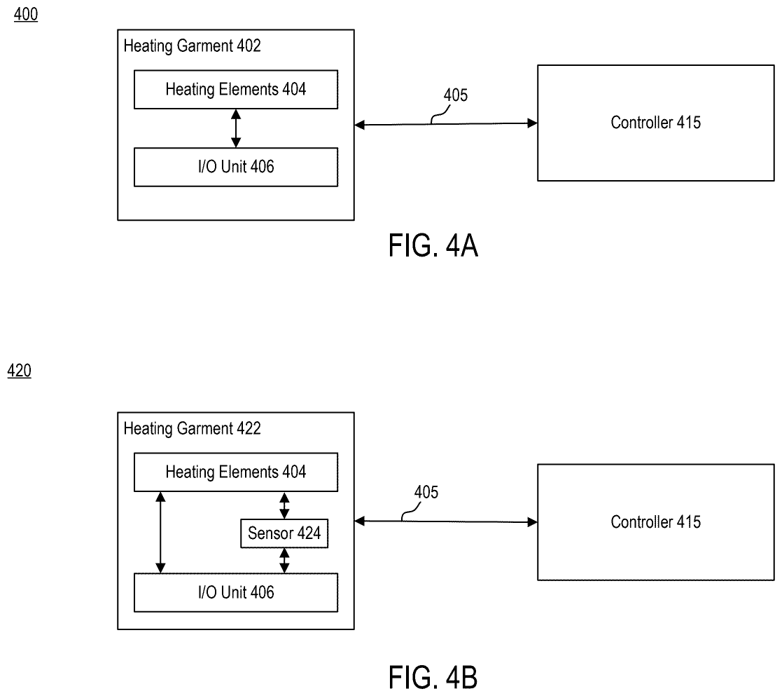

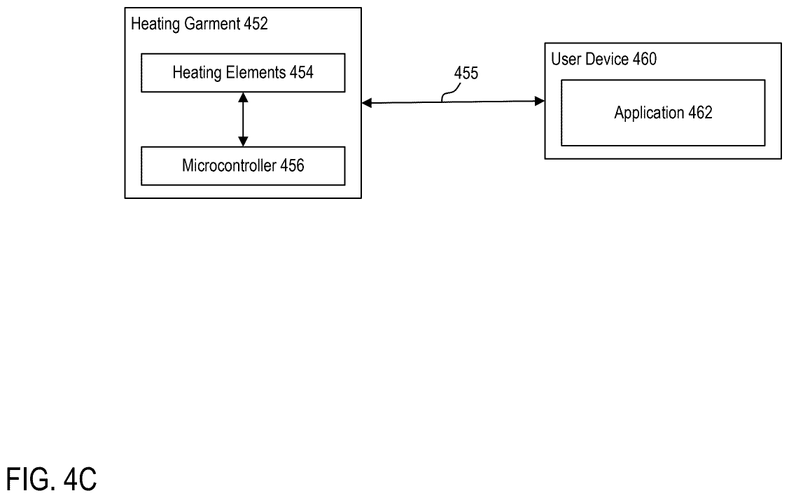

FIG. 4A is a block diagram illustrating components of a heating system 400, according to example embodiments. As shown, heating system 400 includes heating garment 402 and controller 415 communicating via one or more wired or wireless connections 405. Heating garment 402 may be representative of heating garment 100. For example, as shown, heating garment 402 may include heating elements 404 and input/output unit 406. Heating elements 404 may correspond to heating elements 202 illustrated and discussed above in conjunction with FIGS. 2A-2B. In some embodiments, input/output unit 406 may be representative of connection element 108. In some embodiments, controller 415 may be representative of controller 150.

As discussed above, in operation, controller 415 may be coupled with heating garment 402 via input/output unit 406. In some embodiments, controller 415 may be coupled with heating garment 402 via one or more wired connections through input/output unit 406. In some embodiments, controller 415 may be coupled with heating garment via one or more wireless connections 405 (e.g., Bluetooth, WiFi, Zigbee, RFID, NFC, Z-wave, etc.). For example, input/output unit 406 may have an interface configured to receive communications from controller 415 and send communications to controller 415. In operation, for example, a user may utilize controller 415 to increase or decrease the temperature of heating garment 402. Input/output unit 406 may receive this communication from controller 415 and may adjust the amount of current provided to heating elements of heating garment 402, appropriately.

FIG. 4B is a block diagram illustrating components of a heating system 420, according to example embodiments. As shown, heating system 420 includes heating garment 422 and controller 415 communicating via one or more wired or wireless connections 405. Heating garment 422 may be substantially similar to heating garment 402. For example, as shown, heating garment 422 may include heating elements 404 and input/output unit 406. However, heating garment 422 may further include sensor 424. Sensor 424 may be representative of a heating sensor disposed within the pocket or opening of heating garment 422. Sensor 424 may be configured to read or record an operating temperature of heating garment 422. In such embodiments, sensor 424 may allow a user operating controller 415 to set a specific temperature for heating garment 422, akin to setting a temperature of a thermostat. In this manner, input/output unit 406 may be configured to automatically regulate the temperature of heating garment 422 by selectively increasing or decreasing an amount of current provided to heating elements 404.

FIG. 4C is a block diagram illustrating components of a heating system 450, according to example embodiments. As shown, heating system 450 may include heating garment 452 and a user device 460 communicating via one or more communication link 455.

Communication link 455 may be of any suitable type of communication links, including individual connections via the Internet, such as cellular or Wi-Fi networks. In some embodiments, communication link 455 may connect terminals, services, and mobile devices using direct connections, such as radio frequency identification (RFID), near-field communication (NFC), Bluetooth™, low-energy Bluetooth™ (BLE), Wi-Fi™, ZigBee™, ambient backscatter communication (ABC) protocols, USB, WAN, or LAN. Because the information transmitted may be personal or confidential, security concerns may dictate one or more of these types of connection be encrypted or otherwise secured. In some embodiments, however, the information being transmitted may be less personal, and therefore, the network connections may be selected for convenience over security. Communication link 455 may include any type of computer networking arrangement used to exchange data. For example, communication link 455 may be the Internet, a private data network, virtual private network using a public network and/or other suitable connection(s) that enables components in heating system 450 to send and receive information between the components of heating system 450.

Heating garment 452 may be representative of heating garment 100. For example, as shown, heating garment 452 may include heating elements 454 and microcontroller 456. Heating elements 454 may correspond to heating elements 202 illustrated and discussed above in conjunction with FIGS. 2A-2B. In some embodiments, microcontroller 456 may function to take the place of or be in addition to connection element 108. Microcontroller 456 may be configured to control the amount of current provided to heating elements 454 to increase and/or decrease the operating temperature of heating garment 452. Microcontroller 456 may further be configured to communicate wirelessly with user device 460.

User device 460 may be operated by a user, such as, for example, a wearer of heating garment 452. User device 460 may be representative of a mobile device, a tablet, a desktop computer, or any computing system having the capabilities described herein. User device 460 may include an application 462 executing thereon. Application 462 may be representative of an application configured to interface with heating garment 452 via microcontroller 456. Via application 462, a user may be configured to control the operating temperature of heating garment 452. In some embodiments, a user may increase or decrease the operating temperatures similar to how the user would increase or decrease the operating temperature using any of the controllers disclosed herein. In some embodiments, a user may be configured to set an operating temperature to be maintained by microcontroller 456. In some embodiments, application 462 may be a standalone application, such as a mobile application, tablet application, desktop application, or, more generally, a software application. In some embodiments, application 462 may be representative of a web browser configured to communicate with heating garment 452, such that an end user may gain access to microcontroller 456 via a web browser. More generally, application 462 may be configured to provide an interface between user device 460 and heating garment 452 for the purpose of controlling an operating temperature of heating garment 452.

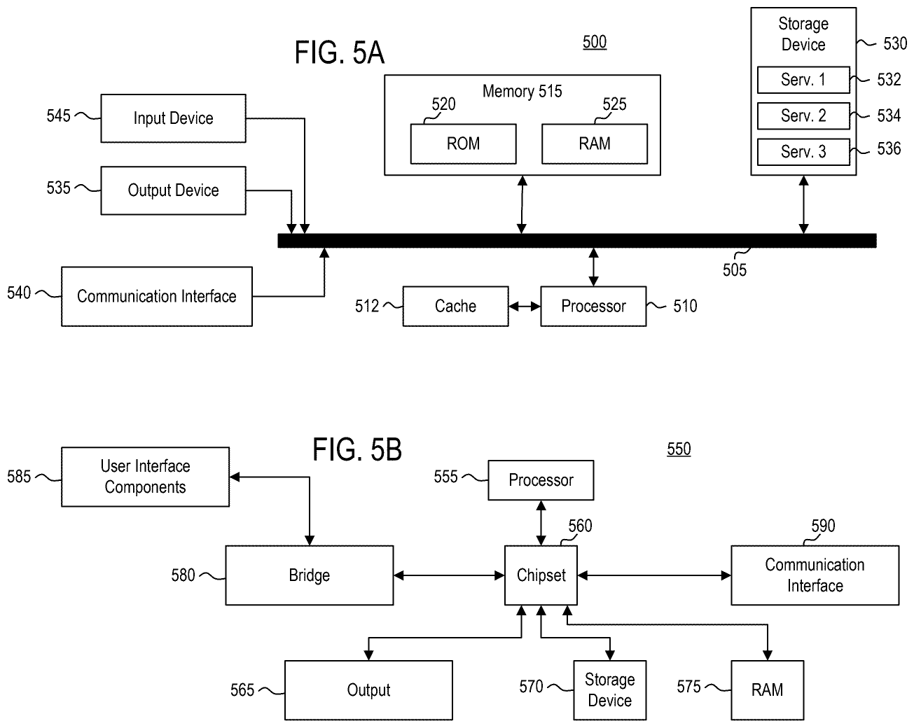

FIG. 5A illustrates a system bus architecture of computing system 500, according to example embodiments. System 500 may be representative of at least user device 460, microcontroller 456, or any of the controllers discussed herein. One or more components of system 500 may be in electrical communication with each other using a bus 505. System 500 may include a processing unit (CPU or processor) 510 and a system bus 505 that couples various system components including the system memory 515, such as read only memory (ROM) 520 and random-access memory (RAM) 525, to processor 510.

System 500 may include a cache of high-speed memory connected directly with, in close proximity to, or integrated as part of processor 510. System 500 may copy data from memory 515 and/or storage device 530 to cache 512 for quick access by processor 510. In this way, cache 512 may provide a performance boost that avoids processor 510 delays while waiting for data. These and other modules may control or be configured to control processor 510 to perform various actions. Other system memory 515 may be available for use as well. Memory 515 may include multiple different types of memory with different performance characteristics. Processor 510 may include any general-purpose processor and a hardware module or software module, such as service 1 532, service 2 534, and service 3 536 stored in storage device 530, configured to control processor 510 as well as a special-purpose processor where software instructions are incorporated into the actual processor design. Processor 510 may essentially be a completely self-contained computing system, containing multiple cores or processors, a bus, memory controller, cache, etc. A multi-core processor may be symmetric or asymmetric.

To enable user interaction with the computing system 500, an input device 545 may represent any number of input mechanisms, such as a microphone for speech, a touch-sensitive screen for gesture or graphical input, keyboard, mouse, motion input, speech and so forth. An output device 535 may also be one or more of a number of output mechanisms known to those of skill in the art. In some instances, multimodal systems may enable a user to provide multiple types of input to communicate with computing system 500. Communications interface 540 may generally govern and manage the user input and system output. There is no restriction on operating on any particular hardware arrangement and therefore the basic features here may easily be substituted for improved hardware or firmware arrangements as they are developed.

Storage device 530 may be a non-volatile memory and may be a hard disk or other types of computer readable media which may store data that are accessible by a computer, such as magnetic cassettes, flash memory cards, solid state memory devices, digital versatile disks, cartridges, random access memories (RAMs) 525, read only memory (ROM) 520, and hybrids thereof.

Storage device 530 may include services 532, 534, and 536 for controlling the processor 510. Other hardware or software modules are contemplated. Storage device 530 may be connected to system bus 505. In one aspect, a hardware module that performs a particular function may include the software component stored in a computer-readable medium in connection with the necessary hardware components, such as processor 510, bus 505, output device 535 (e.g., display), and so forth, to carry out the function.

FIG. 5B illustrates a computer system 550 having a chipset architecture that may represent at least user device 460 or any of the controllers discussed herein. Computer system 550 may be an example of computer hardware, software, and firmware that may be used to implement the disclosed technology. System 550 may include a processor 555, representative of any number of physically and/or logically distinct resources capable of executing software, firmware, and hardware configured to perform identified computations. Processor 555 may communicate with a chipset 560 that may control input to and output from processor 555.

In this example, chipset 560 outputs information to output 565, such as a display, and may read and write information to storage device 570, which may include magnetic media, and solid-state media, for example. Chipset 560 may also read data from and write data to storage device 575 (e.g., RAM). A bridge 580 for interfacing with a variety of user interface components 585 may be provided for interfacing with chipset 560. Such user interface components 585 may include a keyboard, a microphone, touch detection and processing circuitry, a pointing device, such as a mouse, and so on. In general, inputs to system 550 may come from any of a variety of sources, machine generated and/or human generated.

Chipset 560 may also interface with one or more communication interfaces 590 that may have different physical interfaces. Such communication interfaces may include interfaces for wired and wireless local area networks, for broadband wireless networks, as well as personal area networks. Some applications of the methods for generating, displaying, and using the GUI disclosed herein may include receiving ordered datasets over the physical interface or be generated by the machine itself by processor 555 analyzing data stored in storage device 570 or storage device 575. Further, the machine may receive inputs from a user through user interface components 585 and execute appropriate functions, such as browsing functions by interpreting these inputs using processor 555.

It may be appreciated that example systems 500 and 550 may have more than one processor 510 or be part of a group or cluster of computing devices networked together to provide greater processing capability.

While the foregoing is directed to embodiments described herein, other and further embodiments may be devised without departing from the basic scope thereof. For example, aspects of the present disclosure may be implemented in hardware or software or a combination of hardware and software. One embodiment described herein may be implemented as a program product for use with a computer system. The program(s) of the program product define functions of the embodiments (including the methods described herein) and may be contained on a variety of computer-readable storage media. Illustrative computer-readable storage media include, but are not limited to: (i) non-writable storage media (e.g., read-only memory (ROM) devices within a computer, such as CD-ROM disks readably by a CD-ROM drive, flash memory, ROM chips, or any type of solid-state non-volatile memory) on which information is permanently stored; and (ii) writable storage media (e.g., floppy disks within a diskette drive or hard-disk drive or any type of solid state random-access memory) on which alterable information is stored. Such computer-readable storage media, when carrying computer-readable instructions that direct the functions of the disclosed embodiments, are embodiments of the present disclosure.

It will be appreciated to those skilled in the art that the preceding examples are exemplary and not limiting. It is intended that all permutations, enhancements, equivalents, and improvements thereto are apparent to those skilled in the art upon a reading of the specification and a study of the drawings are included within the true spirit and scope of the present disclosure. It is therefore intended that the following appended claims include all such modifications, permutations, and equivalents as fall within the true spirit and scope of these teachings.

1. A heating garment, comprising:
a first piece of material coupled with a second piece of material, wherein a pocket is defined between the first piece of material and the second piece of material;
a plurality of heating elements disposed in the pocket defined between the first piece of material and the second piece of material, the plurality of heating elements configured to run from a top of the heating garment to a bottom of the heating garment; and
a plurality of coupling mechanisms formed on the heating garment, wherein the plurality of coupling mechanisms comprises a first coupling mechanism formed on a first external surface of the first piece of material, a second coupling mechanism formed on a second external surface of the second piece of material, and a third coupling mechanism formed on the second external surface of the second piece of material,
wherein the heating garment is configured to operate between an uncoupled mode and a coupled mode, wherein, in the coupled mode, the second coupling mechanism and the third coupling mechanism are configured to mate with the first coupling mechanism, forming a torso opening and two leg openings in the heating garment.

2. The heating garment of claim 1, further comprising:
a connection element positioned on one of the first external surface or the second external surface, the connection element configured to interface with the plurality of heating elements.

3. The heating garment of claim 2, wherein the connection element comprises an input port configured to interface with a controller.

4. The heating garment of claim 3, wherein the controller is configured to control an operating temperature of the heating garment by increasing or decreasing an amount of current provided to the plurality of heating elements.

5. The heating garment of claim 1, further comprising:
a microcontroller disposed in the pocket formed between the first piece of material and the second piece of material, the microcontroller configured to interface with a client device to control an operating temperature of the heating garment.

6. The heating garment of claim 1, wherein one of the first piece of material or the second piece of material comprises one or more additional pockets for receiving material to increase a weight of the heating garment.

7. The heating garment of claim 1, further comprising:
a temperature sensor disposed in the pocket.

8. The heating garment of claim 1, wherein the plurality of heating elements is disposed in a serpent-like design.

9. The heating garment of claim 1, wherein the first piece of material is welded to the second piece of material.

10. The heating garment of claim 1, further comprising:
a hydrophobic or waterproof material disposed between the first piece of material, the second piece of material, and the plurality of heating elements to protect the plurality of heating elements.

11. A heating system comprising, comprising:
a heating garment comprising:
a first piece of material coupled with a second piece of material, wherein a pocket is defined between the first piece of material and the second piece of material,
a plurality of heating elements disposed in the pocket defined between the first piece of material and the second piece of material, the plurality of heating elements configured to run from a top of the heating garment to a bottom of the heating garment, and
a plurality of coupling mechanisms formed on the heating garment, wherein the plurality of coupling mechanisms comprises a first coupling mechanism formed on a first external surface of the first piece of material, a second coupling mechanism formed on a second external surface of the second piece of material, and a third coupling mechanism formed on the second external surface of the second piece of material,
wherein the heating garment is configured to operate between an uncoupled mode and a coupled mode, wherein, in the coupled mode, the second coupling mechanism and the third coupling mechanism are configured to mate with the first coupling mechanism, forming a torso opening and two leg openings in the heating garment; and
a controller in communication with the heating garment, the controller configured to control an operating temperature of the heating garment.

12. The heating system of claim 11, further comprising:
a connection element positioned on one of the first external surface or the second external surface, the connection element configured to interface with the plurality of heating elements.

13. The heating system of claim 12, wherein the connection element comprises an input port configured to interface with the controller.

14. The heating system of claim 11, wherein the controller is configured to control an operating temperature of the heating garment by increasing or decreasing an amount of current provided to the plurality of heating elements.

15. The heating system of claim 11, further comprising:
a microcontroller disposed in the pocket formed between the first piece of material and the second piece of material, the microcontroller configured to interface with the controller to control an operating temperature of the heating garment.

16. The heating system of claim 15, wherein the controller is a client device.

17. The heating system of claim 11, further comprising:
a temperature sensor disposed in the pocket.

18. The heating system of claim 11, wherein the plurality of heating elements is disposed in a serpent-like design.

19. The heating system of claim 11, wherein the first piece of material is welded to the second piece of material.

20. The heating system of claim 11, further comprising:
a hydrophobic or waterproof material disposed between the first piece of material, the second piece of material, and the plurality of heating elements to protect the plurality of heating elements.
